(19) United States
(12) Patent Application Publication
(10) Pub. No.: US 2014/0074143 A1
(43) Pub. Date: Mar. 13, 2014

(54) INSERTION TOOL AND INSERTION METHOD FOR ARTERIAL TAMPONADE DEVICE

(71) Applicants: Patrick J. Fitzgerald, Poway, CA (US); R. Todd McKinney, Rancho Santa Fe, CA (US); John A. Simpson, Carlsbad, CA (US); Ronald B. Glatts, San Marcos, CA (US)

(72) Inventors: Patrick J. Fitzgerald, Poway, CA (US); R. Todd McKinney, Rancho Santa Fe, CA (US); John A. Simpson, Carlsbad, CA (US); Ronald B. Glatts, San Marcos, CA (US)

(73) Assignee: Sinocclusive LLC, Rancho Santa Fe, CA (US)

(21) Appl. No.: 13/607,543

(22) Filed: Sep. 7, 2012

(51) Int. Cl. A61B 17/10

(52) U.S. Cl. 606/199

(57) ABSTRACT

An ergonomic insertion tool for insertion and retrieval of an arterial tamponade device which has an elongate connecting strut of resiliently deformable material and an arterial compressor at one end. The tool has a handle with a hand grip portion including an actuating trigger, and an elongate barrel. The barrel has a shaft and an outer sleeve telescopically engaged over the shaft. In a first position, a hooked distal end of the shaft protrudes from the sleeve to engage a bend between adjacent end portions of the connecting strut. On movement into a second position, the sleeve extends over the hooked end and compresses the deformable connecting strut into a deformed, U-shaped configuration to hold the device during insertion or retrieval. On movement back into the first position, the deformed device is released to spring out towards the expanded condition.

### BACKGROUND

1. Field of the Invention

The present invention relates generally to tamponade devices used to block or restrict blood flow, and is particularly concerned with an insertion tool for inserting an arterial tamponade device into a body or cavity such as the nasal cavity at a location for reducing bleeding by applying force to one or more blood vessels to partially or completely occlude blood flow.

2. Related Art

Bleeding during a surgical procedure is a problem for the surgeon because it limits visibility, complicates the surgery and can add to overall procedural time. This is particularly true in endonasal surgeries, both due to the fact that the nasal cavity is already confined, and the fact that the nasal cavity is highly vascular and small amounts of bleeding obscure the surgical field. Current standards to reduce bleeding during surgery include: a) topical and local administrated medications, b) controlled hypotension, c) use of pre-operative medications, and d) atraumatic surgical techniques. Post-operative bleeding is also often controlled by nasal packing with absorbable and non-absorbable materials. Despite these measures, intra-operative hemostasis remains a significant challenge.

The sphenopalatine artery, an indirect branch of the external carotid artery, is responsible for the majority of blood flow to the nasal cavity and sinus tissues.

There is therefore a need for a mechanism to reduce or eliminate intra-operative bleeding through the sphenopalatine artery, particularly during endonasal surgery.

### SUMMARY

Embodiments described herein provide for an insertion tool or introducer device for inserting a tamponade device into a body cavity to reduce bleeding during and after surgery or to reduce bleeding as a result of other causes.

According to one embodiment, an insertion tool is provided for insertion or retrieval of a tamponade device which has a flexible, deformable connecting strut and is designed to be inserted into a body cavity in a folded state and which is configured to be expanded after insertion so that opposite ends of the device engage and apply pressure to opposite areas of the body cavity. One end of the device is an arterial compressor which may be an arterial pressure pad while the opposite end may be a substantially flat for engaging an opposite area of the body cavity. When properly positioned, the tamponade device is designed to apply pressure to a blood vessel in a cavity wall with the arterial compressor while the opposite end bears against an opposing portion of the cavity. The arterial compressor is positioned to apply pressure to an area of the body cavity which includes one or more arteries or other blood vessels, so as to occlude the vessel or vessels and reduce or eliminate blood flow through the occluded blood vessel or vessels. This can be useful during surgery to reduce bleeding in the surgical field, after surgery to reduce post-operative bleeding, or to reduce bleeding as a result of trauma or various medical conditions.

The insertion tool basically comprises a handle having a hand grip portion with at least one actuating trigger, and an elongate barrel extending from the handle. The barrel has a shaft and an outer sleeve telescopically engaged over the shaft. The shaft and sleeve are relatively movable between a first position in which a distal end of the shaft protrudes from the sleeve to release or capture a tamponade device and a second position in which the shaft is inside the sleeve with its distal end spaced inwardly from the distal end of the sleeve to capture a collapsed or compressed tamponade device. The distal ends of the sleeve and shaft have co-operating portions designed to engage and retain a bend between first and second end portions of the deformable connecting strut in a folded or partially folded condition during movement from the second to the first position in an insertion procedure or movement from the first to the second position in a retrieval procedure.

In one embodiment, the inner shaft is fixed to the handle and the outer sleeve is movable relative to the shaft back and forth between the first and second positions, and is adjustably connected to the actuating trigger such that movement of the trigger in the same direction moves the outer sleeve from the second to the first position or from the first to the second position. A manually operable reversing lever or selector may be provided to control the direction of movement of the outer sleeve on actuation of the trigger.

The inner shaft and outer sleeve may be of substantially matching rectangular cross section. In one embodiment, a hook portion at the distal end of the shaft is configured to engage the bend in the deformable connecting strut of a tamponade device when the strut in its folded or partially folded condition and the shaft and sleeve are in the first position, and the distal end of the sleeve is configured to extend over the hook portion to retain the bend in the strut as the sleeve moves between the first and second position. The strut is then compressed into a deformed, U-shaped configuration as the distal end of the shaft is moved into the sleeve or the sleeve is extended over the distal end of the shaft and the tamponade device. The spacing between the distal ends of the shaft and sleeve in the second position is arranged so that the majority of the opposite end portions of the strut on each side of the bend or fold are retained inside the sleeve with the pads alone extending from the distal tip of the sleeve in the second position. This makes it easy to hold the device during preliminary insertion into a cavity such as the nasal cavity. The first and second ends of the tamponade device which protrude from the distal tip of the sleeve face outwards and away from one another during insertion.

The barrel is of relatively small cross-sectional dimensions less than the size of the opening into a body cavity such as the nasal cavity, to allow inserting at least the distal end of the barrel holding the generally U-shaped tamponade device into a body or nasal cavity with the arterial compressor at one end of the bent strut oriented to face towards a predetermined region of the nasal cavity wall including the sphenopalatine artery (SPA). The barrel is inserted to a predetermined location in which the oppositely directed ends of the device are substantially aligned with the desired locations for pressure application. The sleeve is then withdrawn to release tamponade device from the insertion tool by retracting the outer sleeve from the distal tip of the inner shaft until the hooked end portion is exposed and the hooked central region of the strut is exposed. During withdrawal of the sleeve, the strut gradually expands outwards from the generally U-shaped deformed configuration towards a fully expanded condition and the opposite ends of the strut engage and press against opposing wall regions of the body cavity before the strut is fully expanded. As the sleeve is withdrawn or retracted, the distal end of the fixed inner shaft can be kept at the same or substantially the same location with the opposite ends of the device aligned with the desired contact locations as the sleeve moves from the second to the first position. Once the distal end of the shaft is exposed and the tamponade device is positioned so that the arterial compressor is pressing against the predetermined region of the cavity wall including the blood vessel and the opposite end or support pad presses against an opposing wall region, the hooked end of the inner shaft can be unhooked from the bend in the strut, and the barrel can be withdrawn from the cavity.

The foregoing procedure may be reversed in order to remove a previously placed tamponade device from a body cavity such as the nasal cavity. First, the relatively movable sleeve and shaft of the barrel are adjusted into the first position, so that the distal tip of the shaft including the hooked distal end portion protrudes from the sleeve. The distal tip of the shaft is then inserted into the cavity and moved into position with the hooked portion aligned with the bend in the previously placed tamponade device. The hooked end portion is then hooked onto the bend in the strut. At this point, the sleeve may be partially advanced until a tongue at the distal end of the sleeve extends over the hooked bend in the tamponade device to hold the device, after which the barrel may be retracted slightly, and the sleeve is further extended to engage over the tamponade device and urge the splayed or deployed end portions of the strut inwardly, moving the ends of the device inwardly and away from the respective opposite wall portions of the cavity. The barrel can then be completely withdrawn, removing the tamponade device from the cavity. This avoids the need to withdraw the device while it is still partially expanded.

The body cavity in which the device is inserted may be a nasal cavity and the barrel dimensions are then selected so that the tip of the barrel can be readily inserted and withdrawn from a typical nasal cavity. Once placed in position, the non-slip texture of the pads, combined with the spring force applied by the flexible legs or end portions of the device, supports the pads in position while occluding or at least partially occluding the underlying artery.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a perspective view of an insertion tool or introducer device for inserting the nasal arterial tamponade device of FIGS. 5 and 6 at a selected position in a nasal cavity, with the insertion tool shown in a first position holding the tamponade device in a retracted, compressed condition;

FIG. 2 is an exploded view of the components of the insertion tool of FIG. 1;

FIG. 3 is a side elevation view of the insertion tool in a second, device release position for release or retrieval of the tamponade device, with the handle broken away to reveal the components of the retraction and release mechanism;

FIG. 4 is a broken away side elevation view similar to FIG. 3, with the tool in the first, device retaining position of FIG. 1;

FIG. 5 is a plan view of an intranasal arterial tamponade device for insertion in a right nasal cavity using the insertion tool of FIGS. 1 to 4, with the device shown in a relaxed, expanded condition;

FIG. 6 is an exploded view of the separated parts of a left nasal cavity version of the tamponade device of FIG. 1;

FIG. 7 is an enlarged, perspective view of the tip of the insertion tool of FIGS. 1 to 4 in the device retaining position of FIGS. 1 and 4, holding the tamponade device in a retracted, collapsed or compressed position prior to insertion in a nasal cavity;

FIG. 8 is a view similar to FIG. 7 but illustrating the outer sheath of the introducer partially retracted and the tamponade device starting to expand;

FIG. 9 is a view similar to FIGS. 7 and 8 but illustrating the device release position of FIG. 3B, in which the outer sleeve is fully retracted with the tamponade device fully expanded and ready to be released;

FIG. 10 is a view similar to FIGS. 7 to 9 but illustrating the tamponade device separated from the insertion tool either after insertion or immediately prior to retrieval; and

FIG. 11 is a coronal cross-sectional view through a nasal cavity illustrating placement of the tamponade device of FIGS. 5 and 6 by the insertion or introducer tool of FIGS. 1 to 4 and 7 to 10, in order to occlude the sphenopalatine artery.

### DETAILED DESCRIPTION

Certain embodiments as disclosed herein provide for an insertion tool or introducer for insertion and placement of an arterial tamponade device between opposing areas of a body cavity so as to apply pressure against a predetermined area of tissue in a wall of the cavity including an artery. In one embodiment, the arterial tamponade device is a human nasal arterial tamponade device and has an arterial compressor designed to apply pressure to the sphenopalatine artery supplying blood to the nose one or more blood vessels, so as to temporarily occlude the artery and stop or reduce blood flow through the artery.

After reading this description it will become apparent to one skilled in the art how to implement the invention in various alternative embodiments and alternative applications. However, although various embodiments of the present invention will be described herein, it is understood that these embodiments are presented by way of example only, and not limitation.

Although the following description and the accompanying drawings are directed to an insertion tool or introducer of suitable configuration and dimensions for placement of an intranasal arterial tamponade device which is configured for placement in a left or right nostril for exerting pressure on the sphenopalatine artery (SPA) which supplies blood to the nose, it will be understood that the device may be configured for placement of tamponade devices of appropriate shape and dimensions for placement in other parts of the body or body cavities to apply pressure against blood vessels in tissue, such as cavity walls, so as to temporarily occlude the vessel or vessels or at least reduce blood flow through the vessel and reduce bleeding as a result of surgery, trauma or the like. Additionally, although the description and drawings illustrate the introducer holding an arterial tamponade device having an arterial compressor at one end which comprises a molded pad and a support at the opposite end comprising a second pad, the introducer may alternatively be used to insert other tamponade devices which have different arterial compressor and support structures including structures integrally formed with the connecting strut.

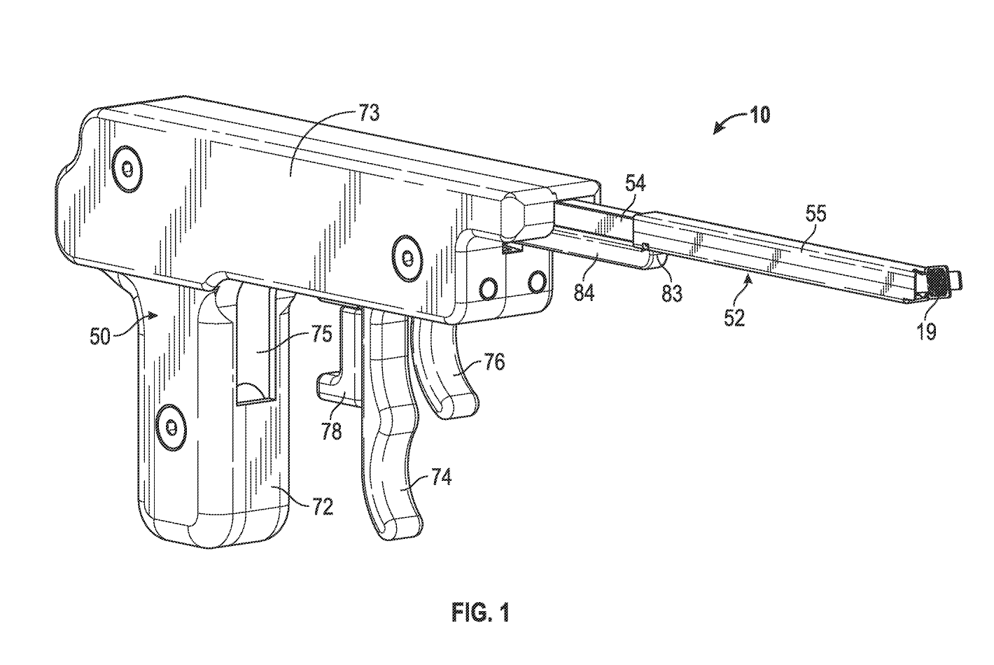

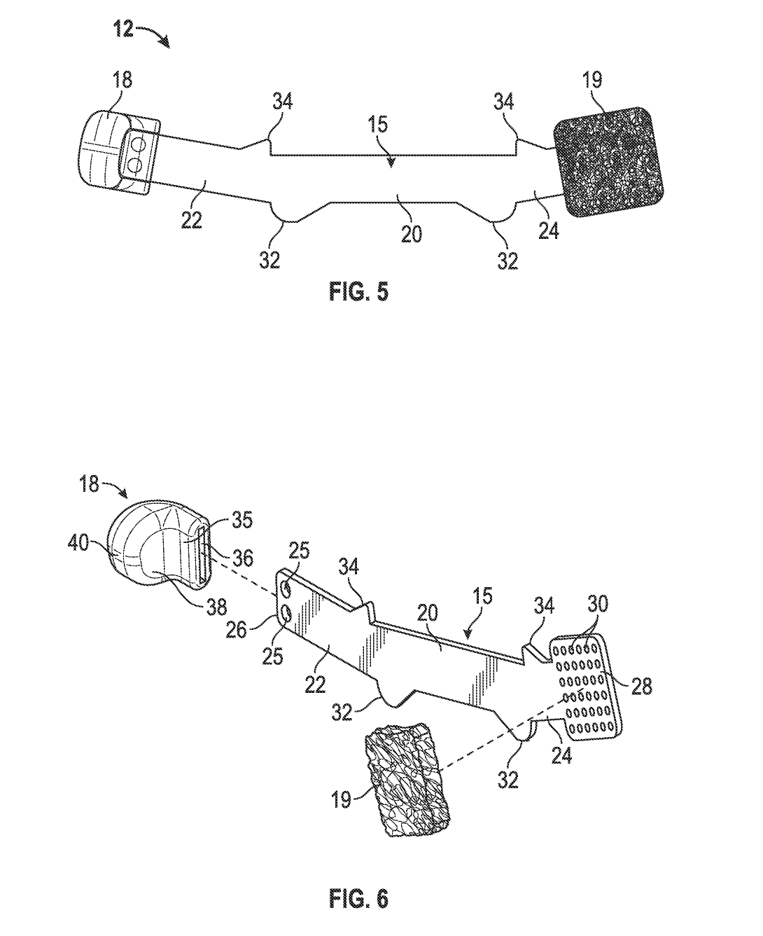

FIGS. 1 to 4 and 7 to 10 illustrate one embodiment of an insertion tool or introducer 10 configured for placement of a nasal arterial tamponade device, such as device 12 as illustrated in FIGS. 5 and 6, at a predetermined position in a nasal cavity to temporarily occlude one or more blood vessels, particularly the sphenopalatine artery in the left nasal cavity, as illustrated in FIG. 11. FIG. 5 illustrates one embodiment of a right nasal cavity device configured for placement in a right nasal cavity, and the corresponding left nasal cavity device is a mirror image of the device 12 illustrated in FIG. 5. FIG. 6 is an exploded view of the components of a left nasal cavity device. The left and right nasal arterial tamponade devices are described in more detail in the co-pending United States patent application of Fitzgerald et al. entitled Arterial Tamponade Device filed on even date herewith, which claims priority from co-pending U.S. provisional Pat. App. No. 61/539,053 filed on Sep. 26, 2011, and the entire contents of both of the foregoing applications are incorporated herein by reference. The components of the left and right nasal cavity devices are identical, but the pads face in opposite directions in the right and left devices. As noted above, although the device illustrated in the drawings has pads at each end, the arterial compressor and support at opposite ends of the device may be of different structure in alternative embodiments, and may be formed integrally with connecting member or strip 15.

Device 12 basically comprises a relatively thin elongate member or flat strip 15 of resilient material which may be a shape memory material with superelastic properties, with an arterial compressor or pressure applying pad 18 secured to one end and a support or second pressure pad 19 at the opposite end. Member 15 may be formed from a length of a suitable springy or resilient metal or plastic wire, rod, or flat ribbon or strip of a material which is biocompatible with body tissues, for example a shape memory alloy material such as nitinol or the like. The elongate member may be solid or tubular.

As illustrated in FIG. 5, the right nasal cavity device when viewed with the end portions upswept and the pads facing the viewer has the arterial pressure pad on the left, while the left nasal cavity device if viewed in the same orientation has the arterial pressure pad on the right. The left and right cavity tamponade devices are formed from the same components as in FIG. 6, with FIG. 6 illustrating the separated components positioned for forming the left cavity tamponade device. As illustrated, strip 15 has a central portion or main body 20 and first and second end portions 22, 24 which are each angled relative to central portion 20. The angled end portions help in visualization when inserting the device and also allow for a larger range of different nasal anatomies. The angle of each end portion relative to the main portion may be in the range from zero to 45 degrees, and in one embodiment the angle was around 10 degrees. A pair of holes 25 extend through the strip adjacent the first end 26 of the strip. The second end portion is enlarged to provide a rectangular or square mounting area or plate 28 for the second pressure pad 19. Multiple holes 30 extend through the second end portion in area 28 to provide for better adhesion of the pressure pad 19. As illustrated in FIG. 6, the pressure pad 19 is a relatively flat, rectangular or square pad of spongy material and has dimensions substantially matching those of area 28. In one embodiment the dimensions are of the order of 0.24 by 0.24 inches. Pad 19 and the corresponding mounting area 28 of strip 15 may be of different peripheral shapes in alternative embodiments, such as circular, oval, rectangular, polygonal, or irregular shapes.

A first pair of guides or tabs 32 project from one side edge of central portion 20 adjacent the respective angled end portions 22, 24, while a second pair of guides or stops 34 project from the opposite side of strip 15 in the respective angled portions, adjacent the junction with central portion 20. The guides 32 and 34 help to place and align the device properly in introducer or insertion tool 10 for placing the device in a nasal cavity.

In one embodiment, the length of member or strip 15 is around 1.5 inches. The distance between the inner edges 37 of tabs or stops 34 is around 0.7 inches, and the length of end portion 22 from edge 37 is around 0.42 inches. The length of end portion 24 from edge 37 up to the end of square area 28 is around 0.38 inches. The width of the central portion 20 and end portion 22 is around 0.14 inches. The thickness of the strip is around 0.016 inches. As noted above, the strip 15 may be of a highly resilient or shape memory material such as Nitinol or similar alloy material.

Device 12 is designed to be collapsed from its original, relaxed or fully expanded shape as illustrated in FIG. 1 into a compact, folded configuration for installation purposes, so that it can be moved to a selected region in a body cavity without significantly contacting structures in the path to the desired region. When constructed of a shape memory material such as nitinol, folding the device is facilitated by cooling to or below its transformation temperature such that the softer martensitic phase is present. The legs deploy towards the extended, unstressed position of FIG. 5 when released from the insertion tool or introducer 10, as described in more detail below, and engage opposite wall regions of the body cavity while the device is still partially folded, so that the partially folded elongate member 15 applies a biasing force pressing the opposite ends or pads 18, 19 against the opposite wall regions of a nasal cavity as illustrated in FIG. 11, with the spring force in the folded elongate member or strip 15 urging the opposing ends or pads 18, 19 against predetermined opposing areas of the nasal cavity. The device is positioned so that the bulbous end portion of pressure pad 18 is urged against predetermined area 23 of the lateral nasal wall including the sphenopalatine artery 12 and foot or pad 19 bears against an opposing area of the nasal septum 45, with one or both of the legs extending over the inferior turbinate 46 and foot 18 engaged in or near the sphenopalatine foramen against the sphenopalatine artery and any of its branches.

The pressure pad or SPA pad 18 which is designed to engage the tissue surface including one or more blood vessels such as the sphenopalatine artery (or one or more blood vessels in a different body cavity in alternative embodiments) is suitably configured to apply sufficient pressure over an area of the artery to at least substantially occlude blood flow in the artery, and also to minimize trauma to the tissue. Pad 18 may be positioned to occlude blood flow in one or more arteries or blood vessels, particularly the sphenoplatine artery and branches of that artery. In the illustrated embodiment, the angled, bulbous end portion 40 is designed to reach better into the SPA foramen and more easily seek a settled and stable position, as indicated in FIG. 11. The tissue contacting surface 40 is rounded or bulbous for better pressure application. The flat septal pad 19 presses against an opposing portion of the septal wall 45, as indicated in FIG. 11, when the device is properly positioned and released from an insertion tool. The septal pad 19 is designed to resist slippage, aid in the delivery of necessary force, assist in holding the device in Position, and potentially limit blood flow to the posterior septum.

The distance between the sphenopalatine artery and the septum in typical individuals is approximately 1.2-1.8 cm, with an average of 1.5 cm. The variability is on the septum side and to a lesser extent the skull size of the patient. The dimensions of device 10 in the fully expanded, relaxed condition of FIG. 5 are such that, when positioned and released at the desired location in nasal cavity 14 as in FIG. 11, the device cannot fully expand and the member 15 is still partially compressed or deformed inwardly from its original straight or partially curved shape so as to apply spring force against the opposing cavity surfaces via pads or feet 18 and 19. The device may be of varying lengths. The fully expanded spacing between the tissue engaging surfaces or pads at the ends or feet of the device is of the order of 30 to 40 mm (1.18 to 1.57 inches), and in one embodiment the fully expanded spacing is around 30 mm (1.18 inches). In one embodiment for use in sphenopalatine artery tamponade, device 10 is designed so that foot 18 applies pressure of around 2000 gm per sq. cm (around 28 psi) or more to the opposing tissue surface of the lateral nasal wall. In some cases, the device may be designed to apply a significantly higher amount of pressure. Devices of different sizes may be provided for different size nasal cavities or for use in different body cavities. The device is designed to be of relatively low profile when installed in the selected position in the nose. The angled offset between the central portion 20 of the connecting member and the ends of the member on which the pads are mounted provides for better visualization of the ends as the compressed device is inserted into a nasal cavity, and thus easier and more accurate placement of the device.

When placed as illustrated in FIG. 11, the pressure pads 18, 19 engage and apply pressure against the nasal mucosa and bone. The device is of relatively low profile when installed and is placed low in the nose out of the normal surgical field. The angled, bulbous end of pressure pad 18 is configured to exert pressure on one or more blood vessels in the area, particularly the underlying sphenopalatine artery 13, so as to temporarily occlude the artery and cut off blood flow to the nasal cavity. This artery is a major blood supply to the nose and occluding it temporarily, for example during endoscopic sinonasal surgeries, reduces intra-operative bleeding and, more importantly, improve visualization during such procedures. At the conclusion of surgery, the device 10 can be removed and discarded, or may be left in place for a time period after surgery if needed.

Delivery and removal of a temporary nasal arterial tamponade device (NATD) is a critical element for the procedure to improve the surgical field during endoscopic endonasal surgery. As a key point of control for the surgeon, the insertion tool or introducer must be easily maneuvered and precisely actuated to accurately and effectively insert, position and release the NATD in the nasal space with minimal difficulty and time, and then easily regain control of and remove the NATD at the end of its use. To accomplish each of these actions, there are a number of important design features included in insertion tool 10.

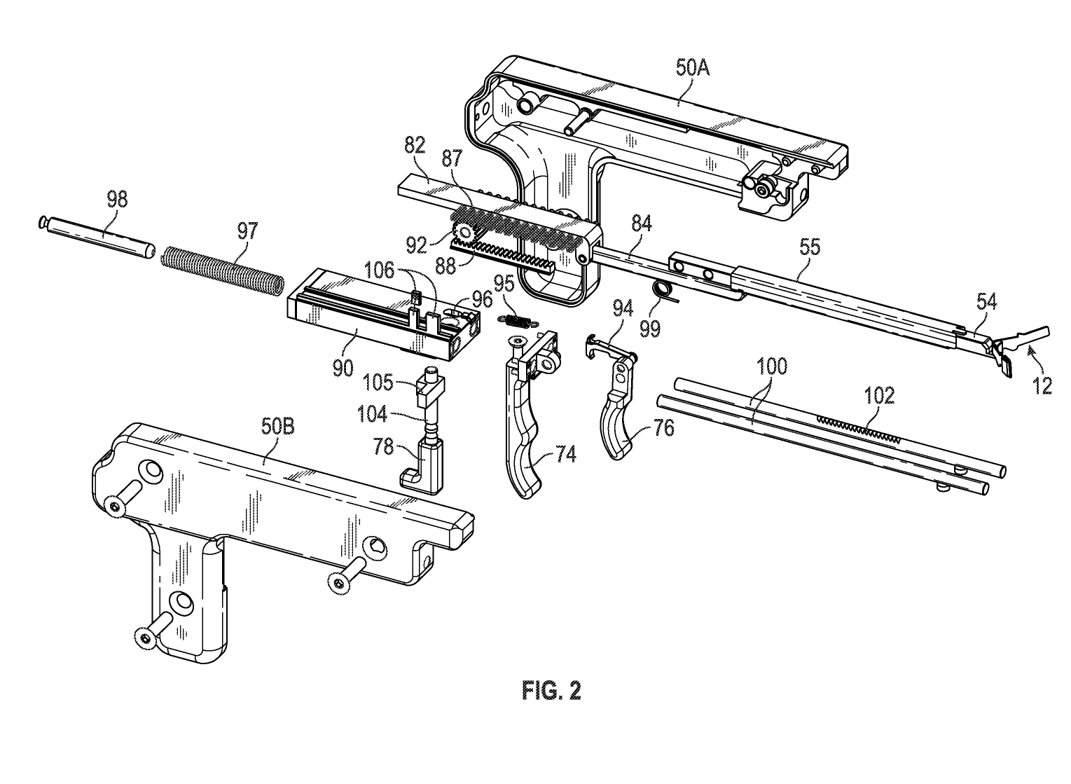

As illustrated in FIGS. 1 to 4, introducer tool 10 has a handle 50 and a barrel 52 extending from handle 50. Barrel 52 comprises telescopically engaged first and second parts which are movable between a first, device retaining position and a second, device releasing or capturing position. In one embodiment, the first part comprises an inner, fixed shaft 54 of rigid or semi-rigid material and the second part comprises an outer sleeve 55 which is movable between an extended position corresponding to the first, device retaining position, as illustrated in FIGS. 1, 4 and 7, and a second, fully retracted, device release position as illustrated in FIGS. 2, 3, 8 and 9 in which the tip 56 of shaft 54 is extended out of sleeve 55 in the second, device release or capture position of the tool. The outer sleeve is of generally rectangular cross section and the shaft is of smaller rectangular dimensions to allow the sleeve to slide telescopically back and forth over shaft 54. The outer cross-sectional dimensions of the sleeve and inner shaft 54 are sufficiently small to allow the tip of shaft 54 to enter and navigate the primary nasal passage, as described in more detail below. In one embodiment, the sleeve 55 has a square cross sectional shape with sides of 0.25 inches in length, but alternative dimensions may be used in other embodiments based on the size of the tamponade device and the size of the cavity or passageway in which the tip of the tool is to be inserted. The 0.25 inch square profile is suitable for the adult nasal cavity. The length of the sleeve 55 in this embodiment may be of the order of 3.0 to 3.5 inches.

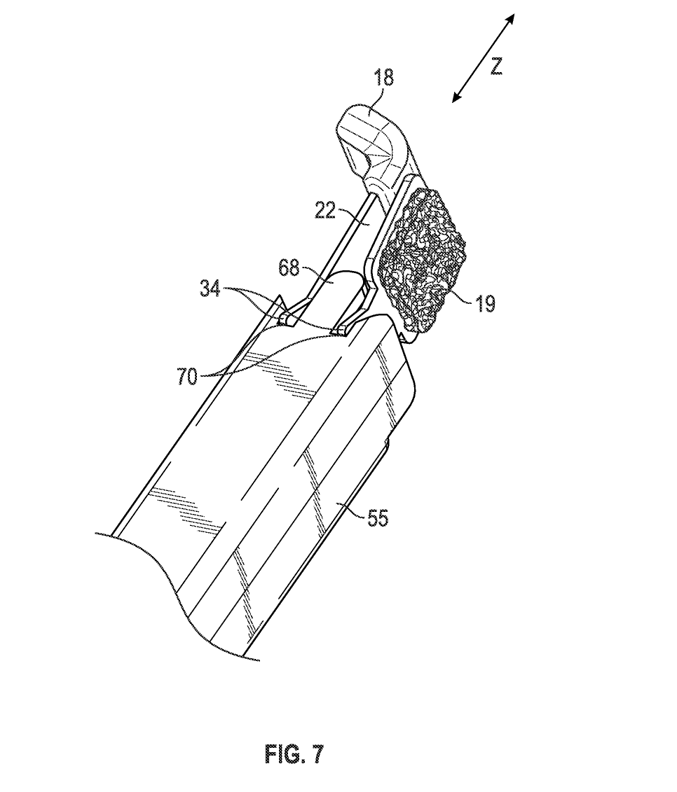

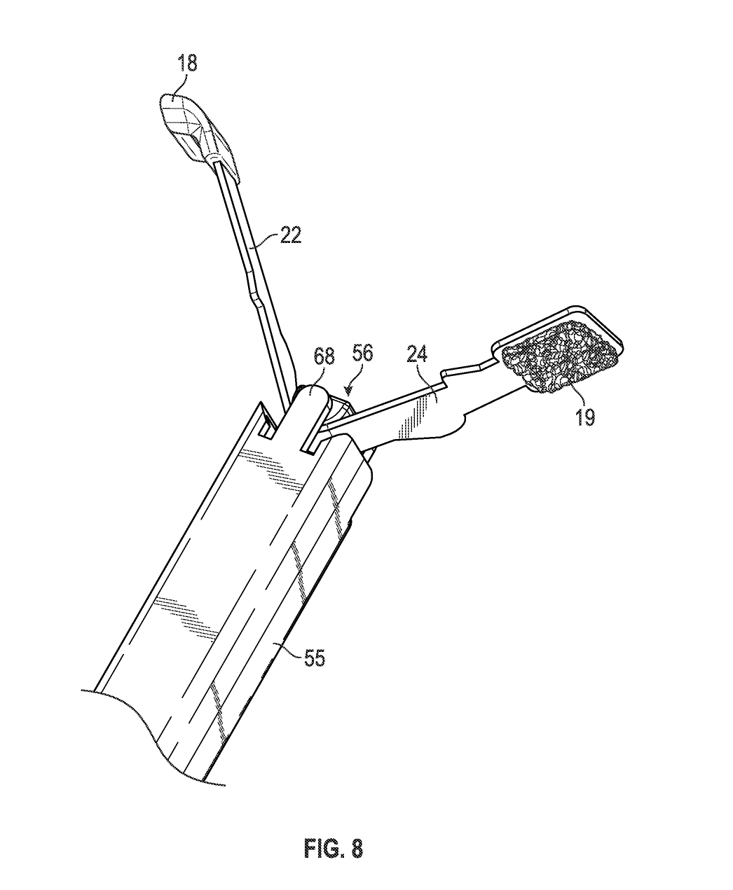

A hook portion at the tip 56 of shaft 54 forms a recess 58 for receiving a bent or folded portion 60 of NATD device 12 between guide tabs 92 when the opposite ends of the device are urged towards one another so that the device adopts a compressed; U or V-like shape. As illustrated in FIG. 7, the hook portion at tip 56 is of predetermined configuration to guide the captured portion of the elongate member 15 into a rounded shape, and has an arcuate guide wall 62 configured for engaging and guiding the arcuate, bent portion 60 of device 12 or a corresponding tamponade device for engaging the left nasal cavity. The radiused wall 62 at the distal end of shaft 54 is designed to access and control the tamponade device and create a more gradual bend or fold as outer sleeve 55 is advanced over the distal tip of shaft. This action folds the NATD to minimize its profile and enable insertion into the nasal cavity.

The outer sleeve contains some unique features to enable control and manipulation of the NATD during delivery and extraction. Specifically, as best illustrated in FIGS. 7 to 10, the top wall of the outer sleeve has a tongue-like feature 68 projecting from the forward end with indents 70 on each side which are designed to engage guide tabs 34 on the upper edge 66 of device 12 when the sleeve 55 is in the fully extended position of FIGS. 1, 4 and 7. The rounded guide tabs 32 on the opposite edge of strip 15 allow the folded strip to slide easily into the sleeve on extension of the sleeve. Tongue-like feature 68 is designed to allow the insertion tool or introducer to maintain capture and control of the NATD 12 after the outer sleeve is retracted and the NATD is preliminarily released. This allows for easy re-capture and re-folding of the NATD in situations where repositioning is necessary.

As illustrated in FIGS. 1 to 4, insertion tool handle 50 is of an ergonomic design which can be readily gripped by the physician's left or right hand. The handle 50 may be made of any suitable robust metal, plastic or other material that can be sterilized for relatively unlimited reuse, or may be made of a disposable material for single use. As illustrated, the handle has a main gripping portion 72, a first, larger control trigger or lever 74 which can be gripped and moved towards recess 75 in main gripping portion to actuate sleeve 55 as described below, and a smaller, distal control trigger or lever 76 which controls fine movement of the sleeve. The larger, proximal lever controls gross movement of the outer sleeve, and the smaller, distal trigger controls fine movement of same. This dual control allows for a 1:1 control movement between the large trigger and the actuating sleeve, and a smaller 6:1 control movement between the small trigger and the sleeve is provided in one embodiment for fine, gradual release or retraction of the NATD 12 as it is positioned into place or removed. Proximal to both triggers is a forward/reverse selector or switch 78 that is moved up or down to reverse the action of both triggers on the outer sleeve.

An actuating mechanism 80 seen in FIGS. 2 to 4 inside the upper part 73 of handle 50 links movement of trigger 74 or 76 to gross or fine advancing or retracting motion of sleeve 55. Although the actuating mechanism is of a rack and pinion type in the illustrated embodiment, other types of actuating linkage or mechanisms between the triggers and sleeve 55 may be used in alternative embodiments. FIG. 2 is an exploded view of tool 10 and illustrates the components of the actuating mechanism between the triggers and the outer sleeve 55 in more detail. As illustrated in FIG. 2, the handle housing is formed in two parts 50A and 50B which house the actuating mechanism. As illustrated in FIGS. 2 to 4, the actuating mechanism includes an upper gear track 82 incorporating friction springs 87 and having a downwardly facing toothed rack 86, a slider block 90 on which an upwardly facing, lower gear track or toothed rack 88 is slidably mounted, and a rotatably mounted pinion or gear wheel 92 engaging the teeth of each toothed rack as indicated in the drawings, so that the toothed racks move in opposite directions. A link arm 84 links sleeve 55 to the upper gear track 82 via hook 83 which engages an opening in the lower wall of sleeve 55. The outer sleeve 55 can be released from hook 83 and removed from the inner shaft 54 for cleaning/sterilization or other purposes.

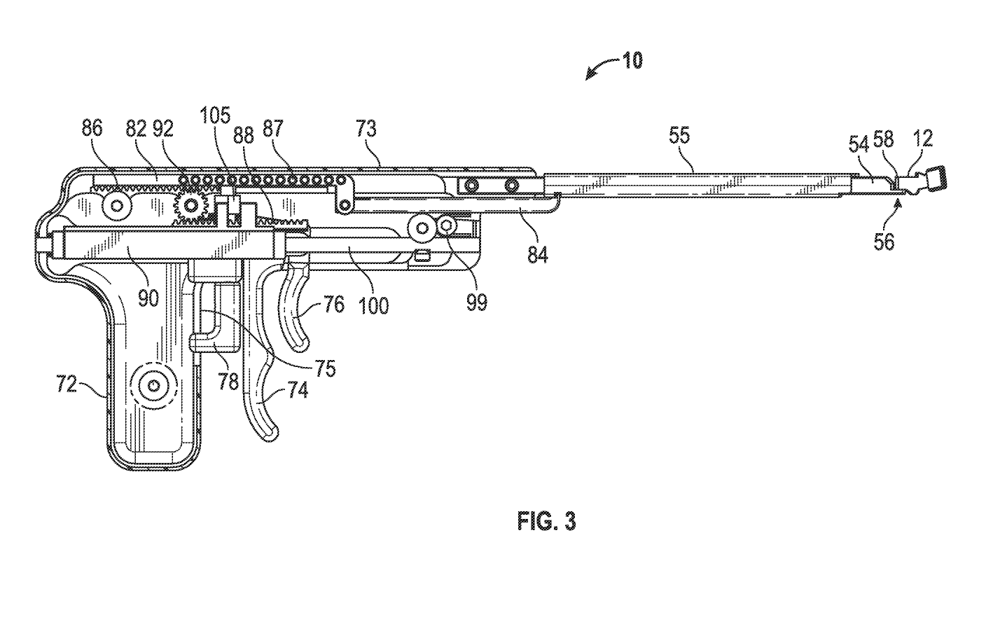

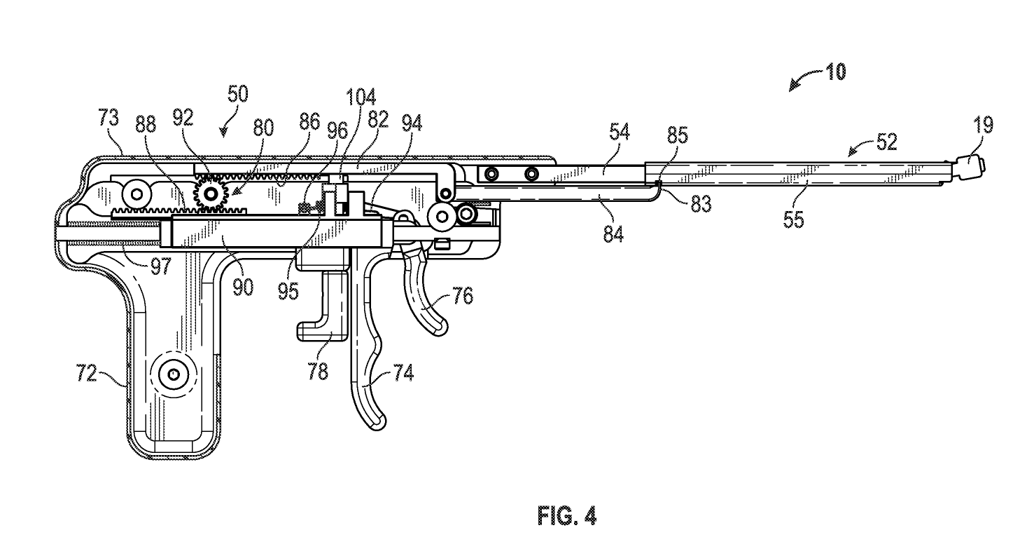

Slider block 90 is slidably mounted on guide shafts 100 which extend through aligned Parallel bores in the block. One of the guide shafts has upwardly facing fine motion control teeth 102, which are exposed through an opening 96 in the upper wall of block 90. Return spring 97 is engaged over guide post 98 secured in an end wall of the handle as seen in FIG. 4, and urges the block 90 towards the extended position of FIG. 4, corresponding to the extended, device retaining position of the sleeve 55. The main control or gross movement trigger 74 is secured to the distal end of slider block 90 at its upper end, so that movement of trigger 74 moves slider block 90. The fine control trigger 76 is pivotally secured to the upper end of main control trigger 74 and connected to ratcheting hook 94 designed to engage fine movement teeth 102 through opening 86 in the slider block. Tension spring 99 is located between link arm 84 and the distal end of guide shaft 100. The forward/reverse selector 78 has an upwardly projecting selector shaft or pin 104 and a link member or block 105 extending transverse to shaft 104 and engaging a slot between upwardly extending guides 106 on slider block 90 (see FIG. 2). Selector 78 is movable between an up/in or raised position as in FIG. 4, in which the upper end of selector shaft 104 engages a groove in the upper gear track 82 adjacent the forward end of upper toothed rack 86, and the block 105 is positioned above the lower gear track or toothed rack 88, and a down/out position (not illustrated) in which the top of the selector shaft is moved down and out of the upper gear track groove, and the block 105 is moved to the lower end of the slot between guides 106 to engage the front end of the lower toothed rack or gear track 88. In the illustrated up/in position of selector shaft 104, pulling of either the main control trigger 74 or the fine control trigger 76 results in pulling of the sleeve inward or rearward, along with upper toothed rack 86, as illustrated for the main control trigger in FIG. 3. In the down/out position of selector 78, engagement of block 105 with the forward end of lower toothed rack or gear track 88 results in movement of this track to the left or rear of the handle or pulling of the main control triggers, and corresponding outward or forward movement of upper toothed rack and gear track 82 in the opposite direction, so that the sleeve 55 is forced outward over shaft 54. If a user wishes to retract the sleeve 55 from the extended or retaining position illustrated in FIG. 4 to the retracted or device release position illustrated in FIG. 3, they squeeze main control trigger 74 towards hand grip 72 with the reversing lever 78 in the up/in position, so upper gear track 82 is urged to the left, retracting sleeve 55 and exposing the tip 56 of shaft 54.

Fine movement of sleeve 55 is controlled by pivotally mounted trigger or lever 76 which is squeezed towards lever 74 to move the sleeve incrementally (6:1 control movement between trigger and sleeve). For fine movement of the sleeve in a direction controlled by selector 78, micro or fine movement trigger 76 is pulled with the index finger towards the main control trigger, while applying pressure to the main control trigger 74 to ratchet the slider block along its guide shaft via ratcheting hook 94. The forward/reverse selector 78 is used to control the direction of fine or incremental movement of the sleeve 55 via fine control trigger 76 in the same manner as the main control trigger 74. If the selector 78 is in the raised position as in FIG. 4, fine movement of the slider block and attached selector 78 along track 102 results in fine movement of the sleeve in the same direction, resulting in an incremental retraction of the sleeve. If the forward/reverse selector 78 is in the lowered position in which it does not engage the upper gear track 82 but the block 105 engages the forward end of toothed rack 88, and the fine control trigger is squeezed, fine movement of the slider block also moves toothed rack 88 to the left, and results in corresponding fine movement of upper toothed rack 86 and linked sleeve 55 to the right or forward over shaft 54. The trigger must be moved through its entire travel in order to engage the next tooth on the guide shaft. Spring 95 urges fine control trigger 76 to the start or extended position illustrated in FIG. 4 when released.

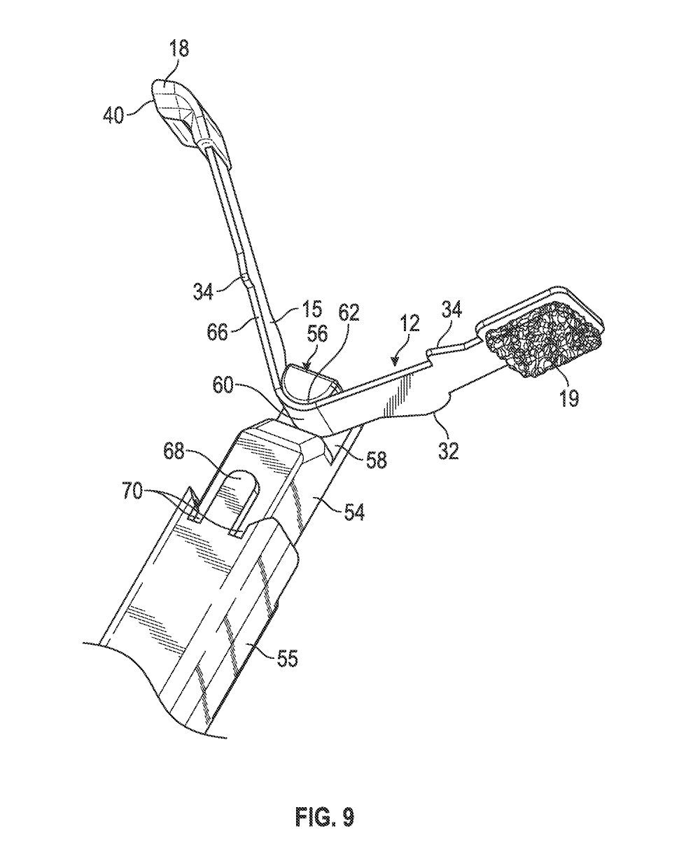

In FIG. 3, sleeve 55 is in the fully retracted position which is also illustrated in FIG. 9. In FIGS. 4 and 7, the sleeve 55 is in the advanced position in which the forward end of the sleeve extends over the hooked end or tip 56 of shaft 54, retracting a hooked tamponade device 12 into the sleeve so that the opposite arms are urged towards one another into a compressed condition, guide tabs 34 engage in indents 70, and tongue 68 extends over the recess 58 in the hooked end to hold the tamponade device in position, as illustrated in FIGS. 1, 4 and 7. In this position, only the ends of the device carrying pads 18 and 19 project out of sleeve 55. The distance between the hooked portion at the distal end of shaft 54 and the distal end of sleeve 55 in the second, device retaining position of FIGS. 4 and 7 is therefore less than the distance from fold or bend 60 to the pads 18 and 19. In one embodiment, this distance may be around 0.45 to 0.5 inches.

FIGS. 7 to 10 illustrate a method for delivery and removal of the right nasal arterial tamponade device 12 of FIGS. 5 and 6 using the insertion tool or introducer 10 of FIGS. 1 to 4. The same method is used for delivery or retrieval of a left nasal arterial tamponade device. As illustrated in FIGS. 1 and 7, the bent connecting strut 15 of device 10 is initially positioned within sleeve 55 in a compressed, deformed state, with a U-shaped bend or fold in connecting strut 15 engaged in the hooked indent at the tip of inner shaft 54 and the opposite legs or end portions 22 and 24 pushed inwardly into a more or less parallel configuration, with the ends carrying pads 18, 19 located outside the sleeve 55. In this position, the sleeve 55 is in the fully advanced or device retaining position extending over the tip 56 of inner shaft 54, with the guide tabs 34 on the upper edge of strut or connecting strip 15 engaging in slots or indents 70 on each side of tongue 68. This is the first, device retention position of the insertion tool. In this position, the tamponade device is held in the compressed condition with only the pads 18, 19 protruding from the tip of the sleeve, so that the device can be more readily inserted into the nasal cavity without contacting wall surfaces or anatomical projections of the cavity. The device retention position for a left nasal arterial tamponade device is similar, except that pads 18 and 19 are reversed when the insertion tool holding the right cavity device 12 as in FIGS. 1 and 7 is inserted into the right nasal cavity. Arterial pad 18 faces to the right when inserted into a right nasal cavity in direction Z, and support pad faces to the left to engage the nasal septum. A corresponding left cavity device is held in the same position prior to insertion into a left nasal cavity, with guide tabs facing upwards, arterial pad 18 facing to the left and support pad 19 facing to the right. Thus, the pads are oriented properly for insertion in the correct nasal cavity.

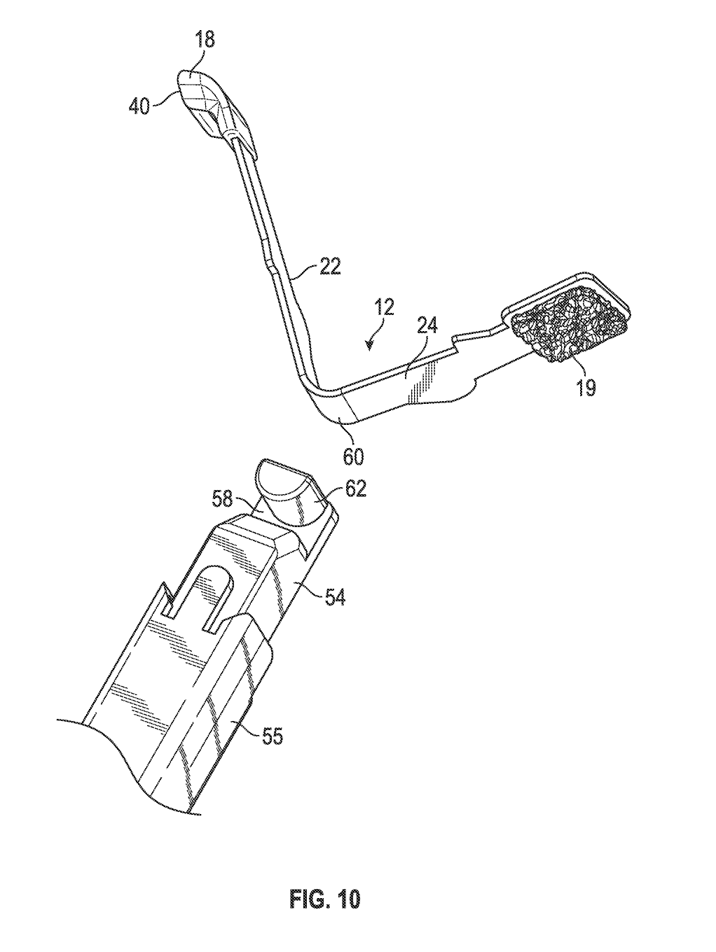

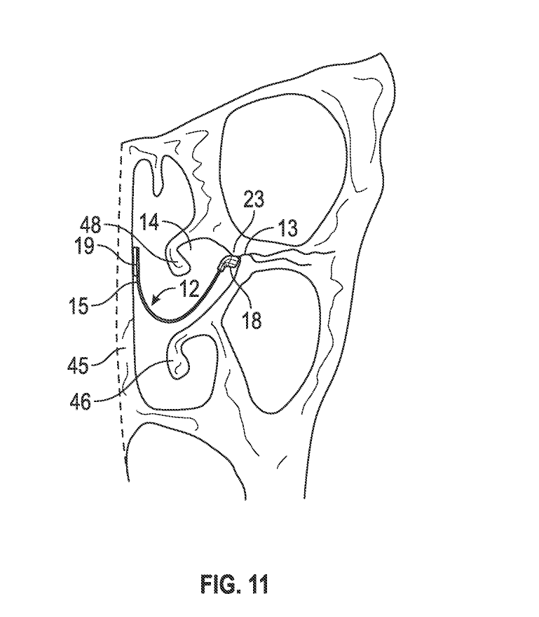

The tip of sleeve 55 is then inserted to a desired location within the nasal cavity in which the opposing pads 18, 19 are aligned or substantially aligned with desired regions of the opposing wall surfaces which they are intended to contact and press against when the device is fully deployed. In other words, arterial pressure pad 18 should be facing the depression in the SPA foramen which houses the SPA, while the opposing support pad 19 faces an opposing portion of the wall of the nasal septum. The physician then engages main trigger 74 with lever 78 in the raised position for retracting sheath 55, and starts to retract the sheath from the position in FIG. 4 towards the fully retracted position of FIG. 3, so as to expose the tip 56 of inner shaft 54, as seen in FIGS. 3 and 9. As soon as the device 12 starts to exit sleeve 54, the end portions 22 and 24 of the device 12 start to spring apart. In FIG. 8, the device 12 is still held in position by tongue 68 extending over the indented or hooked region at the tip 56 of shaft 54 to prevent movement of the bend or fold 60 out of indented region 58. The physician may wish to hold the sleeve in this position as the legs or end portions expand, while adjusting the position of the device 12 to better position the arterial pressure pad for engaging the cavity containing the SPA, with the device in a partially expanded condition. During retraction of the sleeve 54, the physician is able to see the positions of pads 18 and 19 due to the angled end portions 22, 24, which raise the pads above the top wall of sleeve 54 as the device expands. This enables the physician to position the device correctly during insertion, so that the pads 18, 19 are pushed against opposing regions of the nasal cavity wall, specifically against the area or recess 23 of the lateral nasal wall in the middle meatus which includes the sphenopalatine artery and against the opposing region of the nasal septum 26, as illustrated in FIG. 11. Once the device is correctly positioned, the physician retracts the sleeve into the fully retracted position of FIGS. 3 and 9, exposing the hooked end portion 56 of the shaft. The shaft is then separated or unhooked from the fold or bend 60 (FIG. 10), and the tip of the device can be removed from the nasal cavity. The device 12 is designed so that the pressure applied by the device frame or legs against area 23 is sufficient to hold the device in place and to occlude or at least substantially occlude blood flow from the sphenopalatine artery. The roughened consistency of the outer surface of pad 19 helps to hold the pad against slipping once engaged with nasal septum 26.

The procedure is reversed in order to install a tamponade device in the tip of the tool prior to insertion, or in order to retrieve and remove a previously placed device 12 from the nasal cavity after use, for example when occlusion of the sphenopalatine artery is no longer necessary to reduce post-operative bleeding or bleeding following trauma, or to stop or reduce a nose bleed, for example. In order to position a tamponade device prior to installation using tool 10, the physician or assistant simply squeezes main control trigger 74 to retract the sleeve 55 and expose the hooked end of shaft 54, positions the central portion of the device in the hook recess 58, and releases the trigger so that return spring 97 urges the sleeve 55 into the extended position of FIG. 4, withdrawing the device apart from the ends carrying pads 18 and 19 into the compressed condition inside sleeve 55.

In order to retrieve a previously positioned tamponade device 12 from the nose, the tip of the tool is advanced into the nasal cavity with the sleeve retracted as in FIG. 10, and the hooked end 56 is positioned under the fold or bend 60 in connecting strip 12 so that the fold 60 is aligned with recess 58. The tip is then raised so that bend 60 is engaged in recess as in FIG. 9, and the sleeve 55 is extended until tongue 68 covers the hooked end or tip 56 of shaft 54 as in FIG. 8, locking the device in place. Further extension of the sleeve urges the opposite end portions 22, 24 together as the guide tabs 34 engage in slots 70 and the device is retained in the fully folded and retained position of FIG. 7. The tip of the insertion tool may then be gradually withdrawn from the nasal cavity, removing the tamponade device.

Insertion tool or introducer 10 has an ergonomic design, and is easily adapted for use with either of the surgeon's hands. It has a narrow tip that can effectively enter and navigate the primary nasal passage. Shaft 54 can be either rigid or flexible, but is of adequate strength at the tip 56 to grasp and retract the device. Actuation and movement of the sleeve are both predictable and accurate, and accommodate both fine and gross movements. The action is reversible to accommodate both delivery and retraction with the same actuation method

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

We claim:

1. An insertion tool for inserting an arterial tamponade device comprising a flexible, deformable connecting strut having an arterial compressor at one end into a human nasal cavity to apply pressure to a blood vessel in a cavity wall with the arterial compressor while the opposite end bears against an opposing portion of the cavity, comprising:
a handle having a hand grip portion and a body portion;
an elongate barrel extending from the body portion, the barrel having first and second telescopically engaged parts which are relatively movable between a first, extended position and a second, retracted position, the first part being fixed to the body portion and the second part being movable between device retaining and release positions;
a trigger mechanism on the handle configured to move the first part back and forth between the retaining and release positions;
one of the parts comprising a hollow sleeve and the other part comprising an inner shaft, each part having a distal tip, the distal tip of the hollow sleeve extending over the distal tip of the inner shaft in the device retaining position and the distal tip of the inner shaft protruding out of the distal tip of the hollow sleeve in the device release position; and
the distal tip of the shaft having a hook portion configured to engage the bend between adjacent end portions of a flexible, deformable connecting strut of a tamponade device and the hollow sleeve configured to extend over the hook portion and compress the deformable connecting strut into a deformed, U-shaped configuration in the device retaining position;
the distal tip of the sleeve being at a Predetermined distal spacing from the hook portion in the retaining position such that the majority of the deformed, U-shaped connecting strut is within the sleeve and opposite ends of the device protrude from the distal tip of the sleeve.

2. The insertion tool of claim 1, wherein the inner shaft comprises the fixed first part and the outer sleeve comprises the movable second part, the outer sleeve being movable between an extended position corresponding to the device retaining position in which the distal tip of the inner shaft is inside the sleeve and spaced rearward from the distal tip of the sleeve and a retracted position corresponding to the device release position.

3. The insertion tool of claim 1, wherein the trigger mechanism comprises a main control trigger configured for engagement by a user's fingers and movable from a start position in a first direction towards the hand grip portion.

4. The insertion tool of claim 3, further comprising a fine movement control trigger configured for controlling fine movement of the first part of the barrel.

5. The insertion tool of claim 4, further comprising a forward/reverse movement selector movable between first and second positions and configured to reverse the movement of the sleeve as a result of actuation of either the main control trigger or the fine control trigger.

6. The insertion tool of claim 4, wherein the ratio between movement of the main control trigger and the first part of the barrel is 1:1 and the ratio of movement of the fine movement control trigger and the first part of the barrel is 6:1.

7. The insertion tool of claim 4, wherein the main control trigger and fine movement control trigger depend from the body portion of the handle at a location spaced distally from the hand grip portion, the fine movement control trigger being located in front of the main control trigger, and both triggers being configured for engagement and actuation by a user's fingers while gripping the hand grip portion.

8. The insertion tool of claim 3, further comprising a linking mechanism configured to link movement of the control trigger in one direction to bi-directional movement of the first part of the barrel between the device retaining and release positions, and a manually operable forward/reverse movement selector configured to control the direction of movement of the first part, the reversing lever having a first position in which movement of the control trigger in the first direction moves the first part of the barrel in a first direction and a second position in which movement of the fine control trigger in the first direction moves the first part of the barrel in a second direction opposite to the first direction.

9. The insertion tool of claim 1, wherein the outer sleeve and inner shaft are of mating rectangular cross-sectional shape.

10. The insertion tool of claim 1, wherein the hook portion has spaced first and second surfaces defining an indented region configured for receiving the bend between adjacent portions of the connecting strut of the device, at least the second surface being arcuate and configured to substantially match the curvature of the bend in the deformed, U-shaped configuration of the device.

11. The insertion tool of claim 10, wherein the second, arcuate surface is a convex surface located at the distal tip of the shaft and the first surface is spaced rearward from the second, arcuate surface.

12. The insertion tool of claim 1, wherein the distal end of the sleeve has a forwardly extending tongue configured to extend over the hook portion at the distal end of the shaft and retain the hooked bend of the tamponade device in the hook portion at a predetermined position between the release and retaining positions on retrieval or deployment of a device.

13. The insertion tool of claim 12, further comprising a pair of indents on opposite sides of the tongue configured to engage with guide tabs on upper edges of the connecting strut in the retaining position of the barrel parts.

14. The insertion tool of claim 1, wherein the outer sleeve is completely separable from the inner sheath for cleaning and sterilization purposes.

15. A method of deploying an arterial tamponade device into a human nasal cavity in order to temporarily apply pressure to an area of a body cavity wall including a blood vessel and occlude or substantially occlude the vessel to stop or reduce blood flow, comprising:
hooking a central region of an elongate, resilient strut of a tamponade device into a hooked end portion at the distal tip of an inner shaft of a barrel of an insertion tool with the inner shaft protruding from the distal tip of an outer sleeve telescopically engaged over the inner shaft;
extending the distal tip of the outer sleeve over the hook shaped portion to retain the hooked central region of the strut in the hooked end portion;
continuing to move the outer sleeve relative to the inner shaft so that the distal tip of the shaft is retracted into the sleeve and the elongate resilient strut is bent from a substantially straight, relaxed condition into a bent, deformed condition in which opposite ends of the device are moved towards one another into a generally U-shaped, compressed configuration with tissue engaging faces at opposite ends of the strut protruding from the distal tip of the sleeve and facing outwards and away from one another;
inserting at least the distal end of the barrel holding the generally U-shaped tamponade device into a body cavity with an arterial compressor at one end of the bent strut oriented to face towards a predetermined region of the cavity wall which includes a blood vessel;
releasing the tamponade device from the insertion tool at a predetermined location in the body cavity by retracting the outer sleeve from the distal tip of the inner shaft until the hooked end portion is exposed and the hooked central region of the strut is exposed, whereby the strut expands outwards from the generally U-shaped deformed configuration towards a fully expanded condition and the opposite ends of the device engage and press against opposing wall regions of the body cavity before the strut is fully expanded, the arterial compressor configured to press against the predetermined region of the cavity wall including the blood vessel and to apply sufficient pressure to at least partially occlude the blood vessel and reduce blood flow to the body cavity; and
unhooking the hooked end of the inner shaft of the barrel from the central region of the strut and retracting the barrel from the body cavity.

16. The method of claim 15, wherein the body cavity is a nasal cavity and the arterial compressor is pressed against a predetermined region of the lateral nasal wall that includes the sphenopalatine artery in the deployed condition.

17. The method of claim 15, wherein the step of releasing the compressed device comprises allowing the strut to expand until the opposite ends of the device bear against opposing wall regions of the body cavity to hold the device in place.

18. The method of claim 15, further comprising retrieving the tamponade device from the body cavity using the insertion tool after a selected healing period, the step of retrieving the tamponade device comprising inserting the tip of the barrel into the body cavity with the hooked end portion of the inner shaft protruding from the outer sleeve, hooking the hooked end portion over the central bent region of the strut, extending the distal end of the sleeve over the hooked end portion of the shaft to retain the central bent region of the strut, further extending the sleeve over the strut so that the elongate resilient strut is bent from a substantially straight, relaxed condition into a bent, deformed condition in which opposite ends of the device are moved towards one another into a generally U-shaped, compressed configuration and tissue engaging faces of first and second ends move away from the opposing regions of the body cavity, and retracting the barrel and compressed tamponade device from the body cavity.
